SEQUENCE LISTING (43 sequences)

SEQ ID NO: 1 - protein, 20 aa, Artificial Sequence
  Description of Artificial Sequence Synthetic peptide
YDPLQPELDSFKEELDKYFK

SEQ ID NO: 2 - protein, 10 aa, Artificial Sequence
  Description of Artificial Sequence Synthetic peptide
YDPLQPELDS

SEQ ID NO: 3 - protein, 12 aa, Artificial Sequence
  Description of Artificial Sequence Synthetic peptide
DSFKEELDKYFK

SEQ ID NO: 4 - protein, 15 aa, Artificial Sequence
  Description of Artificial Sequence Synthetic peptide
NATRFASVYAWNRKR

SEQ ID NO: 5 - protein, 15 aa, Artificial Sequence
  Description of Artificial Sequence Synthetic peptide
CASYQTQTNSPRRAR

SEQ ID NO: 6 - protein, 15 aa, Artificial Sequence
  Description of Artificial Sequence Synthetic peptide
VGGNYNYLYRLFRKS

SEQ ID NO: 7 - protein, 15 aa, Artificial Sequence
  Description of Artificial Sequence Synthetic peptide
YNYLYRLFRKSNLKP

SEQ ID NO: 8 - protein, 15 aa, Artificial Sequence
  Description of Artificial Sequence Synthetic peptide
VYDPLQPELDSFKEE

SEQ ID NO: 9 - protein, 15 aa, Artificial Sequence
  Description of Artificial Sequence Synthetic peptide
CCSCGSCCKFDEDDS

SEQ ID NO: 10 - protein, 15 aa, Artificial Sequence
  Description of Artificial Sequence Synthetic peptide
AGCLIGAEHVNNSYE

SEQ ID NO: 11 - protein, 15 aa, Artificial Sequence
  Description of Artificial Sequence Synthetic peptide
DPLQPELDSFKEELD

SEQ ID NO: 12 - protein, 15 aa, Artificial Sequence
  Description of Artificial Sequence Synthetic peptide
SCGSCCKFDEDDSEP

SEQ ID NO: 13 - protein, 15 aa, Artificial Sequence
  Description of Artificial Sequence Synthetic peptide
SFTRGVYYPDKVFRS

SEQ ID NO: 14 - protein, 15 aa, Artificial Sequence
  Description of Artificial Sequence Synthetic peptide
GTHWFVTQRNFYEPQ

SEQ ID NO: 15 - protein, 15 aa, Artificial Sequence
  Description of Artificial Sequence Synthetic peptide
DVVIGIVNNTVYDPL

SEQ ID NO: 16 - protein, 15 aa, Artificial Sequence
  Description of Artificial Sequence Synthetic peptide
CLIGAEHVNNSYECD

SEQ ID NO: 17 - protein, 15 aa, Artificial Sequence
  Description of Artificial Sequence Synthetic peptide
NCDVVIGIVNNTVYD

SEQ ID NO: 18 - protein, 25 aa, Artificial Sequence
  Description of Artificial Sequence Synthetic peptide
IDGSSGVVNPVMEPIYDEPTTTTSV

SEQ ID NO: 19 - protein, 10 aa, Artificial Sequence
  Description of Artificial Sequence Synthetic peptide
SGVVNPVMEP

SEQ ID NO: 20 - protein, 18 aa, Artificial Sequence
  Description of Artificial Sequence Synthetic peptide
IHFYSKWYIRVGARKSAP

SEQ ID NO: 21 - protein, 25 aa, Artificial Sequence
  Description of Artificial Sequence Synthetic peptide
KLGSLVVRCSFYEDFLEYHDVRVVL

SEQ ID NO: 22 - protein, 21 aa, Artificial Sequence
  Description of Artificial Sequence Synthetic peptide
MADSNGTITVEELKKLLEQWN

SEQ ID NO: 23 - protein, 11 aa, Artificial Sequence
  Description of Artificial Sequence Synthetic peptide
MADSNGTITVE

SEQ ID NO: 24 - protein, 19 aa, Artificial Sequence
  Description of Artificial Sequence Synthetic peptide
MWLSYFIASFRLFARTRSM

SEQ ID NO: 25 - protein, 20 aa, Artificial Sequence
  Description of Artificial Sequence Synthetic peptide
NAAIVLQLPQGTTLPKGFYA

SEQ ID NO: 26 - protein, 21 aa, Artificial Sequence
  Description of Artificial Sequence Synthetic peptide
HIDAYKTFPPTEPKKDKKKKA

SEQ ID NO: 27 - protein, 15 aa, Artificial Sequence
  Description of Artificial Sequence Synthetic peptide
HYTPSFKKGAKLLHK

SEQ ID NO: 28 - protein, 26 aa, Artificial Sequence
  Description of Artificial Sequence Synthetic peptide
LHKPIVWHVNNATNKATYKPNTWCIR

SEQ ID NO: 29 - protein, 9 aa, Artificial Sequence
  Description of Artificial Sequence Synthetic peptide
CLWSTKPVE

SEQ ID NO: 30 - protein, 20 aa, Artificial Sequence
  Description of Artificial Sequence Synthetic peptide
GIVAIVVTCLAYYFMRFRRA

SEQ ID NO: 31 - protein, 19 aa, Artificial Sequence
  Description of Artificial Sequence Synthetic peptide
KHFYWFFSNYLKRRVVFNG

SEQ ID NO: 32 - protein, 21 aa, Artificial Sequence
  Description of Artificial Sequence Synthetic peptide
GVKHVYQLRARSVSPKLFIRQ

SEQ ID NO: 33 - protein, 11 aa, Artificial Sequence
  Description of Artificial Sequence Synthetic peptide
GYYRRATRRIR

SEQ ID NO: 34 - protein, 18 aa, Artificial Sequence
  Description of Artificial Sequence Synthetic peptide
SSPDDQIGYYRRATRRIR

SEQ ID NO: 35 - protein, 45 aa, Artificial Sequence
  Description of Artificial Sequence Synthetic polypeptide
DAYKTFPPTEPKKDKKKKADETQALPQRQKKQQTVTLLPAADLDD

SEQ ID NO: 36 - protein, 14 aa, Artificial Sequence
  Description of Artificial Sequence Synthetic peptide
SKQLQQSMSSSADS

SEQ ID NO: 37 - protein, 30 aa, Artificial Sequence
  Description of Artificial Sequence Synthetic polypeptide
VNNSYECDIPIGAGICASYQTQTNSPRRAR

SEQ ID NO: 38 - protein, 27 aa, Artificial Sequence
  Description of Artificial Sequence Synthetic peptide
NATRFASVYAWNRKRISNCVADYSLYN

SEQ ID NO: 39 - protein, 36 aa, Artificial Sequence
  Description of Artificial Sequence Synthetic polypeptide
SNNLDSKVGGNYNYLYRLFRKSNLKPFERDISTEIY

SEQ ID NO: 40 - protein, 47 aa, Artificial Sequence
  Description of Artificial Sequence Synthetic polypeptide
LNRALTGIAVEQDKNTEVFAQVKQIYKTPPIKDFGGFNFSQILPDPS

SEQ ID NO: 41 - protein, 13 aa, Artificial Sequence
  Description of Artificial Sequence Synthetic peptide
AYCCNIVNVSLVK

SEQ ID NO: 42 - protein, 19 aa, Artificial Sequence
  Description of Artificial Sequence Synthetic peptide
QIGGYTEKWESGVKDCVVL

SEQ ID NO: 43 - protein, 17 aa, Artificial Sequence
  Description of Artificial Sequence Synthetic peptide
LAVFQSASKIITLKKRW

(19) United States
(12) Patent Application Publication
(10) Pub. No.: US 2023/0364184 A1
(43) Pub. Date: Nov. 16, 2023

(54) RESPIRATORY VIRUS THERAPEUTIC COMPOSITIONS AND METHODS OF PREPARATION AND USE

(71) Applicant: Lawrence LOOMIS, Columbia, MD (US)

(72) Inventor: Lawrence LOOMIS, Columbia, MD (US)

(21) Appl. No.: 18/029,027

(22) PCT Filed: Sep. 29, 2021
(86) PCT No.: PCT/US2021/052640

§ 371 Date: Mar. 28, 2023

Related U.S. Application Data
(60) Provisional application No. 63/085,038, filed on Sep. 29, 2020.

(51) Int. Cl. A61K 38/17; A61K 9/00; A61K 31/7068; A61K 31/403

(52) U.S. Cl. CPC A61K 38/1732; A61K 9/0043; A61K 31/7068; A61K 31/403; B82Y 5/00

(57) ABSTRACT

The invention comprises a therapeutic composition adapted to induce innate and antibody-based immune responses. Embodiments of the present invention relate generally to therapeutic compositions for respiratory viruses suitable for nasal and/or nasopharyngeal administration. More specifically, the therapeutic compositions comprise mannose binding lectins (MBL) including e.g., modified MBL (mMBL), selected viral peptides, and combinations of MBL or mMBL and selected viral peptides, optionally in combination with an adjuvant and/or carrier. The invention provides for the simultaneous stimulation of both the innate and adaptive immune responses as well as the proximal aggregation of the invading respiratory vims to its targeted mucosal antibody which maximizes the opsonization of the infecting pathogen.

### CROSS-REFERENCE TO RELATED APPLICATIONS

The present application is a U.S. National Phase Application under 35 U.S.C. § 371 of International Patent Application No. PCT/US2021/052640 filed Sep. 29, 2021, which claims priority to U.S. Provisional Application No. 63/085,038, filed on Sep. 29, 2020, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 29, 2021, is named 259205_000006_SL.txt and is 13,614 bytes in size.

### BACKGROUND OF THE INVENTION

### Technical Field

Embodiments of the present invention relates generally to therapeutic compositions for respiratory viruses suitable for nasal and/or nasopharyngeal administration. More specifically, the therapeutic compositions comprise mannose binding lectins (MBL) including e.g., modified MBL (mMBL), selected viral peptides, and combinations of MBL or mMBL and selected viral peptides, optionally in combination with an adjuvant and/or carrier.

### Background

Respiratory viruses are responsible for the majority of infectious diseases in humans They are found globally and are generally efficiently transmitted aerobically person to person. Exemplary respiratory viruses include influenza virus, respiratory syncytial virus, corona virus parainfluenza virus, adenovirus and metapneumovirus. Because of their genetic and chemical make-up and easy point of entry into the nasopharynx of the individual, many new respiratory viruses are continually discovered. The use of vaccines administered by intramuscular (IM) injection have had some success in controlling the spread of disease but the problem with viral mutation has necessitated the investigation of alternative methods for challenging the virus. Interestingly, many of the respiratory viruses are glycosylated at critical points on the structure of the virus and lectins which are glycan binding proteins that binds to these glycans. Targeting these glycosylated residues can both aggregate the virus which impedes its ability to be spread aerobically or systemically, as well as interfere with the intracellular transmission of the virus.

A problem that has surfaced with the current use of vaccines administered intramuscularly, is the continued spread of the virus by fully vaccinated individuals, who may now be classified as asymptomatic carriers of the virus. Although vaccines have resulted in good systemic protection from disease, there is little evidence that important neutralizing antibodies IgA1 and IgA2 and IgG are present in the mucosal lining of the nasopharynx or in the saliva of the vaccinated individual. The portal of entry for respiratory viruses is the nasopharynx and an important defense against infection is the production of localized targeted IgA found in the mucosal lining of the nasopharynx. For example, it has been found that early antiviral response in the nose of exposed individuals to SARS-CoV-2 can determine the severity of the COVID-19 infection.

Pandemic SARS-related coronaviruses (S-CoV) have caused worldwide epidemics periodically in past years (e.g., human alpha-corona viruses, SARS1, MERS), with infections ranging from mild respiratory illnesses to those causing major pulmonary complications resulting in severe morbidity and significant numbers of deaths, particularly in the elderly and very young individuals; especially those persons immuno-compromised as well as those with underlying chronic diseases. While a number of vaccines are available for the current pandemic of SARS-CoV-2, there is a pressing need for a quick, effective therapeutic treatment to inhibit the pathologic sequelae of COVID-19 in persons who in large numbers are refusing vaccines or have them unavailable due to insufficient supplies. Given the modeling after Flu vaccinations that play an important role, primarily in controlling and preventing major outbreaks, there is available a therapeutic formulation using neuraminidase therapeutics that are effective current treatments for unvaccinated individuals to supplement flu vaccines that can provide relief for unvaccinated individuals with flu, as well as help thwart such periodic flu pandemics. Currently commercial therapeutic methods for COVID-19 consists of mainly giving the anti-viral inhibitor Veklury (remdesivir), an intravenous nucleotide prodrug, or using specific monoclonal antibodies (Mabs) to block coronavirus entry. Additionally, two oral antiviral drugs to treat COVID-19 are under development. Pfizer is currently developing PF-07321332 and is in phase I trials while Merck is currently developing molnupiravir and is also in phase I clinical trials.

What is needed is a therapeutic composition that comprises peptides that can be selected to be protective against a respiratory virus, e.g., coronavirus, that can inhibit viral infection (e.g., viral entry into cells at the initial point of contact (e.g., the nasopharyngeal area)) but also engender an innate immune response in the subject receiving the therapeutic composition, thereby augmenting the intrinsic host pathways responsible for further immune activation of the host adaptive response that can provide durable immunity for extended periods.

What is needed, therefore, is a therapeutic composition that can induce innate and adaptive antibody-based immune responses, and can preferably opsonize elimination of the target virus. Embodiments of the present invention relates generally to therapeutic compositions for respiratory viruses suitable for nasal and/or nasopharyngeal administration. More specifically, the therapeutic compositions comprise mannose binding lectins (MBL) including e.g., modified MBL (mMBL), selected viral peptides, and combinations of MBL or mMBL and selected viral peptides, optionally in combination with an adjuvant and/or carrier.

### BRIEF SUMMARY OF THE INVENTION

As specified in the Background Section, there is a great need in the art to identify technologies for new respiratory virus therapeutic compositions and use this understanding to develop novel compositions. Embodiments of the present invention relate generally to therapeutic compositions for respiratory viruses suitable for nasal and/or nasopharyngeal administration. More specifically, the therapeutic compositions comprise mannose binding lectins (MBL) including e.g., modified MBL (mMBL), selected viral peptides, and combinations of MBL and/or mMBL and selected viral peptides, optionally in combination with one or more of an adjuvant, a carrier, and an excipient.

In an aspect, the present invention provides a method that simultaneously stimulates both the innate and adaptive immune responses against a respiratory virus and opsonizes its elimination by the proximal aggregation of the invading virus to its targeted mucosal antibody.

In an aspect, the present invention provides a method for treating or preventing SARS-COV-2 comprising administering to a patient in need thereof a therapeutically effective amount of a therapeutic composition comprising a mannose binding lectin (MBL)/modified mannose binding lectin (mMBL) and mixtures thereof, optionally in further combination with one or more of an adjuvant, a carrier, and an excipient.

In another aspect, the present invention provides a method for treating or preventing SARS-COV-2 comprising administering to a patient in need thereof a therapeutically effective amount of a therapeutic composition comprising a mannose binding lectin (MBL)/modified mannose binding lectin (mMBL) and mixtures thereof, in combination with at least one peptide selected from the group consisting of SEQ ID NOs: 1-43, optionally in further combination with one or more of an adjuvant, a carrier, and an excipient.

In another aspect, the present invention provides a method for treating or preventing SARS-COV-2 comprising administering to a patient in need thereof a therapeutically effective amount of a therapeutic composition comprising at least one peptide selected from the group consisting of SEQ ID NOs: 1-43.

These and other objects, features and advantages of the present invention will become more apparent upon reading the following specification in conjunction with the accompanying description, claims and drawings.

### DETAILED DESCRIPTION OF THE INVENTION

There is a great need in the art to identify technologies for new respiratory virus therapeutic compositions and use this understanding to develop novel compositions. Embodiments of the present invention relates generally to therapeutic compositions for respiratory viruses suitable for nasal and/or nasopharyngeal administration. Preferably, these therapeutic compositions can simultaneously stimulate both the innate and adaptive immune responses against a respiratory virus and opsonize its elimination by the proximal aggregation of the invading virus to its targeted mucosal antibody. More specifically, the therapeutic compositions comprise mannose binding lectins (MBL) including e.g., modified MBL (mMBL), selected viral peptides, and combinations of MBL or mMBL and selected viral peptides, optionally in combination with an adjuvant and/or carrier.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm. ”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “MBL/mMBL” encompasses natural/wildtype mannose binding lectins, mannose binding lectin super family proteins, and various modified mannose binding lectins, such as for example and not limitation, FcMBL (IgGFc fused to MBL) as described in U.S. Pat. Nos. 9,150,631; 9,632,085; 10,696,733; 10,526,399; and 10,538,562 and US Patent Publication No. 2018/0371058, all of which are herein incorporated by reference in their entireties.

As used herein, the term “subject” or “patient” refers to mammals and includes, without limitation, human and veterinary animals. In a preferred embodiment, the subject is human.

As used herein, the term “combination” of a therapeutic composition and at least a second pharmaceutically active ingredient means at least two, but any desired combination of compounds can be delivered simultaneously or sequentially (e.g., within a 24 hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. The pharmaceutical carrier can be adapted for use with an oral or nasal dosage form, such as for example and not limitation, a carrier adapted for use in an oil suspension, a spray, a suspension, a solution, an emulsion, a nasal spray, a buccal spray, nasal rinse, nasal cream, nasal gel, mouthwash, rapidly dissolving sublingual tablet and rapidly dissolving thin film. Compositions according to the claimed invention can be contained in, e.g., an atomizer or inhaler, and can be suitable for inhalation, insufflation, and/or nebulization. Suitable pharmaceutical carriers are described in “Remington's Pharmaceutical Sciences” by E. W. Martin. Further, the pharmaceutical carrier can be suitable for nanoparticle or microparticle formulations, e.g., microencapsulated nanoparticle or microparticle formulations.

The term “analog” or “functional analog” refers to a related modified form of a polypeptide, wherein at least one amino acid substitution, deletion, or addition has been made such that said analog retains substantially the same biological activity as the unmodified form, in vivo and/or in vitro.

The term “antibody,” as used herein, refers to an immunoglobulin molecule which specifically binds with an antigen. An antibody can be an intact immunoglobulin derived from a natural source or from a recombinant source. Such antibody can comprise an immunoreactive portion of an intact immunoglobulin. The antibody may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. Any macromolecule, including virtually all proteins or peptides, can serve as an antigen. Furthermore, antigens can be derived from recombinant or genomic DNA. Any DNA which comprises a nucleotide sequences or a partial nucleotide sequence encoding a protein that elicits an immune response therefore encodes an “antigen” as that term is used herein. Furthermore, an antigen need not be encoded solely by a full-length nucleotide sequence of a gene. Partial nucleotide sequences of more than one gene may be used, for example these nucleotide sequences may be arranged in various combinations to elicit a desired immune response. Moreover, an antigen need not be encoded by a “gene” at all. An antigen can be generated synthesized or can be derived from a biological sample.

The term “agent” includes any substance, metabolite, molecule, element, compound, or a combination thereof. It includes, but is not limited to, e.g., protein, oligopeptide, small organic molecule, glycan, polysaccharide, polynucleotide, and the like. It can be a natural product, a synthetic compound, a chemical compound, or a combination of two or more substances. Unless otherwise specified, the terms “agent,” “substance,” and “compound” can be used interchangeably. Further, a “test agent” or “candidate agent” is generally a subject agent for use in an assay of the invention.

The term “binding” refers to a direct association between at least two molecules, due to, for example, covalent, electrostatic, hydrophobic, ionic and/or hydrogen-bond interactions.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components.

As used herein, the term “immune response” includes T-cell mediated and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Parenteral” administration of an immunogenic composition includes, e.g., subcutaneous (s.c.), intravenous (i.v.), intramuscular (i.m.), or intradermal (i.d.) injection, or infusion techniques.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In the context of the present invention, the term “Respiratory Virus” includes but is not limited to influenza virus, respiratory syncytial virus, corona virus, parainfluenza virus, adenovirus and metapneumo virus.

In the context of the present invention, the term “antiviral agent” comprises a therapeutic such as an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), RNA polymerase inhibitors, protease inhibitors, helicase inhibitors, immunomodulators, antisense compounds, short interfering RNAs, short hairpin RNAs, micro RNAs, RNA aptamers, ribozymes, and combinations thereof. Preferably the anti-viral agent is chosen from the group consisting of abacavir, didanosine, emtricitabine, lamivudine, stavudine, tenofovir disoproxil fumarate, zidovudine (AZT), atazanivir, darunavir, fosamprenavir, indinavir, nelfinavir, ritonavir, saquinavir, tipranavir, enfuviritide, maraviroc, dolutegravir, elvitegravir, raltegravir, cobicistat, efavirenz, nevirapine, tocilizumab, EIDD-280, etravirine, molnupravir (EIDD-2801) and PF-07321332. In an embodiment, a combination of antiviral agents may be used. Preferably the combination of anti-viral agents is chosen from the group consisting of (i) rilpivine, abacavir and lamivudine; (ii) abacavir, dolutegravir and lamivudine; (iii) abacavir, lamivudine and zidovudine; (iv) atazanavir and cobicistat; (v) darunavir and cobicistat; (vi) efavirenz, emtricitabine and tenofovir disoproxil fumerate; (vii) elvitegravir, cobicistat, emtricitabine, tenofovir alafenamide fumerate; (viii) elvitegravir, cobicistat, emtricitabine and tenofovir disoproxil fumerate; (ix) emtricitabine, rilpivirine and tenofovir alafenamide; (x) memtricitabine, rilpivirine and tenofovir disoproxil fumerate; (xi) emtricitabine and tenofovir alafenamide; (xii) emtricitabine and tenofovir disoproxil fumerate; (xiii) lamivudine and zidovudine; (xiv) lopinavir, retinovir and interferon-beta 1b; (xv) lopinavir and ritonavir; and (xvi) remdesivir, molnupiravir and PF-07321332. Preferably the anti-viral agent is remdesivir. Preferably the anti-viral agent is favipiravir.

In an embodiment, the antiviral agent for use with therapeutic compositions of the invention comprises: a combination of antibodies against the respiratory virus of interest (e.g., monoclonal antibodies such as casirivimab and imdevimab), remdesivir, AT-527, molnupiravir and/or PF-07321332.

In the context of the present invention, the term “vaccine” (also referred to as an immunogenic composition) refers to a substance that induces anti-respiratory virus immunity or suppresses a respiratory virus upon inoculation into an animal.

Within the meaning of the present invention, the term “conjoint administration” is used to refer to administration of a composition according to the invention and another therapeutic agent simultaneously in one composition, or simultaneously in different compositions, or sequentially (preferably, within a 24 hour period).

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds.(1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Compositions of the Invention

Without wishing to be bound by theory, it is suggested that respiratory viruses can be successfully treated by the administration of a nasal spray form of a therapeutic composition, the therapeutic composition comprising at least two component active ingredients. As a non-limiting example, such a therapeutic formulation suitable for treating and/or preventing the SARS-CoV-2 respiratory virus is described as follows: (i) a mannose binding lectin (MBL) or modified MBL (mMBL) as described herein, (ii) viral peptides specific to SARS-CoV-2 as described herein, and/or (iii) combinations of (i) and (ii), optionally in combination with an adjuvant, carrier, or other excipient. In some embodiments, the therapeutic composition is suitable for nasal and/or nasopharyngeal administration. In some embodiments, the therapeutic composition is present in the form of nanoparticles, such as microencapsulated nanoparticles.

The first component of the formulation, a MBL or mMBL as described herein, is designed to immediately immobilize the virus at the point of entry. This component can minimize the ability of the virus to spread aerobically to others and systemically to the infected subject, thus possibly mitigating severe clinical disease. It is known that the majority of SARS-CoV-2 viral surfaces are heavily glycosylated, in particular with the sugar mannose. This mannose coating is very compatible with the mucosal lining of the nasopharynx, thus enabling the virus to easily populate this entry site. Moreover, the glycosylated surfaces can hide sensitive portions of the virus from the host's immune system. This first component of the treatment is a novel engineered and modified mannose binding lectin (mMBL) which is similar to natural MBL produced by many individuals at low levels in the liver. Natural MBL is part of the innate immune system but it is not ubiquitously produced and individuals lacking sufficient levels are known to be more susceptible to pathogens and respiratory disease. The mMBL can bind to the virus and trigger the lectin/component fixation pathway in the infected subject. Also, the mucosal IgA2 that is found in the mucosal lining is coated with mannose, and the mMBL is able to aggregate with the SARS-CoV-2 virus, thereby activating the host complement fixation system to effectively eliminate the virus. Another major advantage of using mMBL is the elimination of the threat of emerging viral variants and their progressive ability to escape host immunity. The mutations of SARS-CoV-2 are primarily due to single point amino acid substitutions found in the SARS-CoV-2 spike (S) and receptor binding domain (RBD) proteins. These mutations can occur frequently and have led to variants such as the Delta variants which may be more resistant to the antibodies produced by the currently available vaccines. Given that an integral part of the makeup of the virus is its extensive mannose coating and its requirements for its mainstay in the mucosal lining of the nasopharynx, it becomes a potential target for SARS-CoV-2 therapeutics, as variations of the virus eliminating the need for this coating is highly unlikely. In fact, to eliminate all viral glycosylations would not be point mutations, but major structural alterations crippling the virus' existence.

A second component of the therapeutic composition includes viral peptides. These peptides can be used alone or in combination with the mMBLs described herein. These peptides can be selected by analyzing the specific antibody responses found in the saliva and serum of infected individuals to a mapped linear peptide array of the infecting virus. The proposed antiviral treatment can be administered as a nasal or nasopharyngeal treatment at the site of infection.

The second part of the formulation can stimulate the production of targeted localized neutralizing antibodies. This stimulation, for example, can be obtained by using certain specific peptides of the SARS-CoV-2 virus that were found to be associated with the highest IgA responses both in the saliva and serum to SARS-CoV-2 as well as being able to engender long lasting T-cell help. These immunoregulatory peptides are the primary constituents of an adjuvanted mix that can optimize the host targeted IgA response to the virus. The MBL/mMBL constituent of the treatment can aggregate the virus along with the resident IgA, thereby magnifying the opsonization of this complex to eliminate the virus. It can also extend the residence time for such peptides to generate more specific IgA antibodies in the nasopharynx.

Thus, the therapeutic compositions of the invention are capable of simultaneously stimulating both the innate and adaptive immune responses against a respiratory virus and opsonizing its elimination by the proximal aggregation of the invading virus to its targeted mucosal antibody. The aggregation of the virus in the nasopharynx may also improve opsonization.

In an embodiment, the component parts of the treatment (e.g., the viral peptides and/or the MBL/mMBL), can be in the form of nanoparticles such as microencapsulated nanoparticles.

A first aspect of the invention comprises utilizing low amounts of mannose binding lectins (MBL), including modified MBL as discussed herein, that can compete with glycosylated viral antigens (e g , mannose-containing antigens) that are associated with viral pathogens, such as the several known SARS-related pandemic strains, as well as any future SARS-related pandemic viruses or other respiratory viruses with similar characteristics. The MBL/mMBL component may be used alone or in combination with IgA specific antigens that are short peptides or short peptide mimics of select viral (e.g., COVID-19) antigenic sequences (e.g., select “spike”; nucleocapsid, open reading frame (orf)-type protein sequences, as well as other non-structural protein (nsp) viral regions, which are all highly conserved in the known, sequenced pandemic coronaviruses; (SARS-2/CoV-2019; SARS-1, MERS as well as several known Bat coronaviruses; NCBI database: NC_045512.2(SARS2); GCF_00864885.1 (SARS1); GCF_000901155.1(MERS); GCF_000868045.1(Bat-HKU9); MN996532.2(Bat-RaTG13)) that are thought to be precursor mammalian zoonotic hosts. At least one or more of these COVID-19 peptide epitopes are not only to be located regions of the viral particle, such as coronavirus virions or in regions of viruses known to mutate frequently. Suitably these IgA associating viral (e.g., SARS-CoV-2) antigenic epitopes are antibody accessible peptides typically less than 30 amino acid residues, which can be chemically synthesized by standard GMP solid phase synthesis methods known to the art. The formulation of the therapeutic complex may be either the comingling of the viral peptide antigens and the MBL/mMBL protein, optionally in a nanoparticle (e.g., microencapsulated nanoparticle) or each component individually in its own nanoparticle (e.g., microencapsulated nanoparticle).

In another aspect, the invention provides therapeutic compositions comprising respiratory viral antigens that are peptides or peptide mimics of select antigenic IgA binding sequences conserved in the viral structural regions of the known, sequenced pathogenic respiratory viruses, such as those of the beta-coronaviruses (SARS-2/CoV-2019; SARS-1, MERS as well as several known Bat and alpha/beta-coronaviruses). In a non-limiting exemplary embodiment, therapeutic compositions according to the claimed invention can comprise at least one or more SARS-CoV peptide epitopes that are located within the pathogenic SARS-CoV-2 host's receptor binding domains (RBD), within the adjacent cell binding protein domain regions, and/or within the SARS-CoV-2 nucleocapsid or structure surrounding the RNA genome. These regions are not known to mutate frequently. Additional peptides from other regions can be included, such as orf or other non-structural proteins that are predicted to interact with the IgA binding antibody. Still other additional peptides may be located within regions that can mutate frequently, such as the spike (S) protein. Suitably these SARS-CoV-2 antigenic epitopes are non-toxic peptides, chemically synthesized by standard GMP solid phase synthesis methods known to the art, and can be bound covalently with MBL proteins. In an embodiment, the viral peptides are located in one or more of the following regions of SARS-CoV-2: the spike protein (NCBI Reference Sequence: YP_009724390.1); the nucleocapsid protein (GenBank: QHD43423.2); the membrane glycoprotein (GenBank: QHU79207.1); the ORF3A protein (NCBI Reference Sequence: YP_009724391.1); the ORF8 protein (NCBI Reference Sequence: YP_009724396.1); and/or the envelope protein (NCBI Reference Sequence: YP_009724392.1).

In another aspect of the invention, the MBL/mMBL and/or viral peptide composition can include an adjuvant such as, e.g., a mucosal stimulative protein carrier (e.g., toxoidal peptide(s) derived from, but not limited to: tetanus toxoid[TT]; diphtheria toxoid[DT]; pertussis toxoid[PT]; cholera toxoid [CT or Dukoral®]; or mixtures thereof). Without wishing to be bound by theory, it is suggested that such carriers can enable a sufficient and sustained persistence adherence of the therapeutic composition to mucosal surfaces in order to effect useful blockage of viral binding to nasopharyngeal tissue surfaces, and allow for specific IgA targeting antibody (for example, of SARS-CoV-2 protein) viral regions.

In an embodiment, the MBL/mMBL and/or viral peptide composition comprises alkali or alkali earth metal ions. Preferably the alkali earth metal ion is Ca++.

In an embodiment, the MBL/mMBL is an oligomer comprising at least one MBL/mMBL subunit. Preferably the MBL/mMBL is a tetramer, pentamer or hexamer Preferably the MBL/mMBL is a tetramer or hexamer

In another aspect of the invention, the MBL/mMBL component is combined with a peptide component comprising at least three or more peptide antigenic IgA epitope sequences (comprising, for example in SARS-CoV-2, multiple domains and/or comprise epitopes of spike protein domain regions or regions adjacent to SARS-CoV-2 viral structural proteins) to form the therapeutic composition, alone or in further combination with at least one adjuvant, carrier, and/or excipient. Mixing of these polyvalent components with MBL/mMBL may be based on antibody titers against epitope antigens, determined by standard immunological methods known to the art.

In an embodiment, the therapeutic composition comprises between about 1 and about 1000 μg/mL of mMBL, e.g., FcMBL (IgGFc fused to MBL). Preferably the therapeutic composition comprises between about 1 and about 100 μg/mL of mMBL, e.g., FcMBL (IgGFc fused to MBL).

In an embodiment, the therapeutic composition comprises polymeric MBL/mMBL, such as a tetrameric, pentameric, or hexameric form of MBL/mMBL.

In an embodiment, the at least three or more peptide antigenic sequences can comprise one or more epitopes from multiple SARS2-CoV-2 orf and/or non-structural protein (nsp) residues and/or comprise epitopes of non-virion protein domain regions (typically expressed in infected cells). Mixing of these peptide antigenic sequences can be based on antibody titers against epitope antigens, determined by standard immunological methods known to the art.

In an embodiment, the at least three or more peptide antigenic epitope sequences can comprise multiple SARS-CoV-2 virion residues and/or can comprise epitopes of IgA antibody accessible domain virion protein regions (for example, envelope, spike, and/or matrix proteins of the SARS-CoV-2 viruses). Mixing of these peptide antigenic sequences can be based on antibody titers against epitope antigens, determined by standard immunological methods known to the art.

In an embodiment, the therapeutic composition comprises an adjuvant, e.g., an adjuvant that exhibits a mucosal IgA response.

In another aspect, the invention provides a combination of the MBL/mMBL component and/or peptide component in a nanoparticulate vehicle that forms a stable formulation for extended periods of time, with or without added specific adjuvant(s), carrier(s), and/or excipient(s), using standard nanoparticle techniques known to the art. Manganese or calcium phosphate-based adjuvants are commonly used in the art, however non-metallic or nucleotide-based adjuvants can also be used.

In another aspect, the invention provides a method of introducing the therapeutic composition to a subject by different mucosal stimulative routes, known to the art using suitable dosing regimens determined by taking into account the factors well known to the art, including age, weight, sex and medical status of the patient as well as the route chosen for immunogen administration. Without wishing to be bound by theory, it is suggested that therapeutic compositions according to the composition can require no reformulation of peptide components from year to year. It is possible that periodic treatment of the therapeutic compositions can be performed at intervals to be determined by mucosal and serologic titers against antigenic IgA epitopes by standard immunological methods known in the art. Although a preferred method of mucosal stimulation is nasal/nasopharygeal administration via, e.g., a nasal spray or drops or buccal formulation, any other formulation that provides for the nasal, nasopharyngeal, or oral administration of the targeted treatment, such as a mouthwash, gargle, sublingual or buccal film or tablet, or toothpaste, as well as incorporation of the components into a lozenge, candy or chewing gum, is also considered. Additional forms of the therapeutic composition encompassed herein include but are not limited to an oil suspension, a spray, a suspension, a solution, an emulsion, a nasal spray, nasal drops, a buccal spray, nasal rinse, nasal cream, nasal gel, mouthwash, chewing gum, rapidly dissolving sublingual or buccal tablet, rapidly dissolving thin film, and formulations adapted for inhalation, insufflation, and/or nebulization.

In another aspect, the invention provides a method comprising administering therapeutic compositions according to the invention, prevent, delay, reduce, inhibit or otherwise restrict the pathogenic viral infections in clinical applications for such subjects so exposed. More specifically, the invention comprises administering effective dosages of peptide antigens and/or MBL/mMBL as discussed herein, as well as optionally with carriers, adjuvants, and/or excipients, to elicit multiple, protective antibody titers that can afford a sustained prophylactic defense against pandemic or respiratory viruses, such as SARS-CoV-2, beta-coronaviral infections, or other viral pathogens.

The present invention deals with the development of effective prophylactic therapeutic and mucosal-adherent MBL/mMBL and/or peptide compositions that can block the select viral pandemic agent, such as the beta-coronaviruses that have emerged in the past two decades and including the current SARS2-CoV/COVID-19 (S-CoV) viruses and variants as well as the recent SARS1 and MERS viral agents. Compositions according to the invention can activate the subject's innate immune system, via effecting initially the mannose binding lectin antibodies (e.g., IgA) to bind to non-mutated glycopeptides located upon abundant conserved viral epitope surfaces within the virion protein regions that are responsible for binding to the viruses host cellular receptors such as ACE2 and/or neuropilin for entry.

The invention is unique in that it is also based upon the selection of short peptide sequences (with or without mimetic residues) that are known to bind IgA antibodies in highly conserved S-CoV infections and/or are also in the virion protein domain regions adjacent to spike/cell attachment protein specific regions of most all known pathogenic S-CoV type viruses. Normally such short peptide sequences (“epitopes”) are poorly immunogenic and thus unable to stimulate any immune system to mount any significant, protective antibody responses against such short peptide-epitope targets. It is suggested that a nanoparticulated MBL complex is able to adhere stably to nasopharyngeal surfaces where the respiratory viral agent(s) initially enters and thus be able to block access into the host cells. Further, it is suggested that a microencapsulated nanoparticulated complex can improve residence time of the composition in the nasopharynx. By modifying such MBL-linked IgA binding epitope sequences by chemical synthesis coupling to unique short peptide spacers and chemically conjugating these components to a toxoidal carrier protein(s) that renders these peptide epitopes immunostimulatory the resultant therapeutic can affect both innate and adaptive immune responses.

### Specific Therapeutic Compositions of the Invention

1. A therapeutic composition comprising a S-CoV PODTC2_SARS2 ‘spike’ protein peptide, beginning at position 1138, of YDPLQPELDSFKEELDKYFK SEQ ID NO.: 1; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

2. A therapeutic composition comprising a S-CoV PODTC2_SARS2 ‘spike’ protein peptide, beginning at position 1138, of YDPLQPELDS SEQ ID NO.: 2; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

3. A therapeutic composition comprising a S-CoV PODTC2_SARS2 ‘spike’ protein peptide, beginning at position 1146, of DSFKEELDKYFK SEQ ID NO.: 3; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

4. A therapeutic composition comprising a S-CoV PODTC2_SARS2 ‘spike’ protein peptide, beginning at position 343 of NATRFASVYAWNRKR SEQ ID NO.: 4; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

5. A therapeutic composition comprising a S-CoV PODTC2_SARS2 ‘spike’ protein peptide, beginning at position 671 of CASYQTQTNSPRRAR SEQ ID NO.: 5; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

6. A therapeutic composition comprising a S-CoV PODTC2_SARS2 ‘spike’ protein peptide, beginning at position 445 of VGGNYNYLYRLFRKS SEQ ID NO.: 6; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

7. A therapeutic composition comprising a S-CoV PODTC2_SARS2 ‘spike’ protein peptide, beginning at position 449 of YNYLYRLFRKSNLKP SEQ ID NO.: 7; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

8. A therapeutic composition comprising a S-CoV PODTC2_SARS2 ‘spike’ protein peptide, beginning at position 1137 of VYDPLQPELDSFKEE SEQ ID NO.: 8; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

9. A therapeutic composition comprising a S-CoV PODTC2_SARS2 ‘spike’ protein peptide, beginning at position 1247 of CCSCGSCCKFDEDDS SEQ ID NO.: 9; optionally in combination with one or more of MBL/mMBL, an adjuvant, a carrier, an additional therapeutic,and/or an excipient. The composition is optionally formulated for nasal/nasopharyngeal administration. The composition is optionally in the form of a nanoparticulate, such as an encapsulated nanoparticulate.

10. A therapeutic composition comprising a S-CoV PODTC2_SARS2 ‘spike’ protein peptide, beginning at position 647 of AGCLIGAEHVNNSYE SEQ ID NO.: 10; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

11. A therapeutic composition comprising a S-CoV PODTC2_SARS2 ‘spike’ protein peptide, beginning at position 1139 of DPLQPELDSFKEELD SEQ ID NO.: 11; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

12. A therapeutic composition comprising a S-CoV PODTC2_SARS2 ‘spike’ protein peptide, beginning at position 1249 of SCGSCCKFDEDDSEP SEQ ID NO.: 12; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

13. A therapeutic composition comprising a S-CoV CoV PODTC2_SARS2 ‘spike’ protein peptide, beginning at position 31 of SFTRGVYYPDKVFRS SEQ ID NO.: 13; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

14. A therapeutic composition comprising a S-CoV PODTC2_SARS2 ‘spike’ protein peptide, beginning at position 1099 of GTHWFVTQRNFYEPQ SEQ ID NO.: 14; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

15. A therapeutic composition comprising a S-CoV PODTC2_SARS2 ‘spike’ protein peptide, beginning at position 1127 of DVVIGIVNNTVYDPL SEQ ID NO.: 15; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

16. A therapeutic composition comprising a S-CoV CoV PODTC2_SARS2 ‘spike’ protein peptide, beginning at position 649 of CLIGAEHVNNSYECD SEQ ID NO.: 16; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

17. A therapeutic composition comprising a S-CoV PODTC2_SARS2 ‘spike’ protein peptide, beginning at position 1125 of NCDVVIGIVNNTVYD SEQ ID NO.: 17; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

18. A therapeutic composition comprising of a S-CoV PODTC3 ‘ORF3A’ protein peptide, beginning at position 249-273 of IDGSSGVVNPVMEPIYDEPTTTTSV SEQ ID NO.: 18; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

19. A therapeutic composition comprising a S-CoV PODTC3 ‘ORF3A’ protein peptide, beginning at position 253 of SGVVNPVMEP SEQ ID NO.: 19; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

20. A therapeutic composition comprising a S-CoV PODTC8 ‘ORFS’ protein peptide, beginning at position 39-56 of IHFYSKWYIRVGARKSAP SEQ ID NO.: 20; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

21. A therapeutic composition comprising a S-CoV PODTC8 ‘ORFS’ protein peptide, beginning at position 94-118 of KLGSLVVRCSFYEDFLEYHDVRVVL SEQ ID NO.: 21; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

22. A therapeutic composition comprising a S-CoV PODTCS ‘Membrane’ protein peptide, beginning at position 1-21 of MADSNGTITVEELKKLLEQWN SEQ ID NO.: 22; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

23. A therapeutic composition comprising a S-CoV PODTCS ‘Membrane’ protein peptide, beginning at position 1 of MADSNGTITVE SEQ ID NO.: 23; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

24. A therapeutic composition comprising a S-CoV PODTCS ‘Membrane’ protein peptide,: beginning at position 91-109 of MWLSYFIASFRLFARTRSM SEQ ID NO.: 24; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

25. A therapeutic composition comprising a S-CoV PODTC9 ‘Nucleocapsid’ protein peptide, beginning at position 154 of NAAIVLQLPQGTTLPKGFYA SEQ ID NO.: 25; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

26. A therapeutic composition comprising a S-CoV PODTC9 ‘Nucleocapsid’ protein peptide, beginning at position 356-376 of HIDAYKTFPPTEPKKDKKKKA SEQ ID NO.: 26; optionally in combination with one or more of MBL/mMBL, an adjuvant, an additional therapeutic, a carrier, and/or an excipient. The composition is optionally formulated for nasal/nasopharyngeal administration. The composition is optionally in the form of a nanoparticulate, such as an encapsulated nanoparticulate.

27. A therapeutic composition comprising a S-CoV PODTC1 Replicase polyprotein 1a_SARS2′ protein peptide, beginning at position 1974 of HYTPSFKKGAKLLHK SEQ ID NO.: 27; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

28. A therapeutic composition comprising a S-CoV PODTC1 Replicase polyprotein 1a_SARS2′ protein peptide, beginning at position 1986-2011 of LHKPIVWHVNNATNKATYKPNTWCIR SEQ ID NO.: 28; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

29. A therapeutic composition comprising a S-CoV PODTC1 Replicase polyprotein 1a_SARS2′ protein peptide, beginning at position 2012-2020 of CLWSTKPVE SEQ ID NO.: 29; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

30. A therapeutic composition comprising a S-CoV PODTC1 Replicase polyprotein 1a_SARS2′ protein peptide, beginning at position 3051 of GIVAIVVTCLAYYFMRFRRA SEQ ID NO.: 30; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

31. A therapeutic composition comprising of a S-CoV PODTC1 Replicase polyprotein 1a_SARS2′ protein peptide, beginning at position 3651 of KHFYWFFSNYLKRRVVFNG SEQ ID NO.: 31; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

32. A therapeutic composition comprising a S-CoV PODTC7 ORF7A SARS2′ protein peptide, beginning at position 70-90 of GVKHVYQLRARSVSPKLFIRQ SEQ ID NO.: 32; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

33. A therapeutic composition comprising a S-CoV PODTC9 ‘Nucleocapsid’ SARS2′ protein peptide, beginning at position 85 of GYYRRATRRIR SEQ ID NO.: 33 optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

34. A therapeutic composition comprising a S-CoV PODTC9 ‘Nucleocapsid’ SARS2′ protein peptide, beginning at position 78 of SSPDDQIGYYRRATRRIR SEQ ID NO.: 34; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

35. A therapeutic composition comprising of a S-CoV PODTC9 ‘Nucleocapsid’ SARS2′ protein peptide, beginning at position 358-403 of DAYKTFPPTEPKKDKKKKADETQALPQRQKKQQTVTLLPAADLDD SEQ ID NO.: 35; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

36. A therapeutic composition comprising a S-CoV PODTC9 ‘Nucleocapsid’ SARS2′ protein peptide, beginning at position 404 of SKQLQQSMSSSADS SEQ ID NO.: 36; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

37. A therapeutic composition comprising a S-CoV PODTC2_SARS2 ‘spike’ protein peptide, beginning at position 656-686 of VNNSYECDIPIGAGICASYQTQTNSPRRAR SEQ ID NO.: 37; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

38. A therapeutic composition comprising a S-CoV PODTC2_SARS2 ‘spike’ protein peptide, beginning at position 343-371 of NATRFASVYAWNRKRISNCVADYSLYN SEQ ID NO.: 38; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

39. A therapeutic composition comprising a S-CoV PODTC2_SARS2 ‘spike’ protein peptide, beginning at position 438-473 of SNNLDSKVGGNYNYLYRLFRKSNLKPFERDISTEIY SEQ ID NO.: 39; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

40. A therapeutic composition comprising of a S-CoV PODTC2_SARS2 ‘spike’ protein peptide, beginning at position 763-810 of LNRALTGIAVEQDKNTEVFAQVKQIYKTPPIKDFGGFNFSQILPDPS SEQ ID NO.: 40; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

41. A therapeutic composition comprising a S-CoV PODTC4 ‘Envelope’ protein peptide, beginning at position 40 of AYCCNIVNVSLVK SEQ ID NO.: 41; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

42. A therapeutic composition comprising a S-CoV PODTC3 ‘ORF3A’ protein peptide, beginning at position 185-203 of QIGGYTEKWESGVKDCVVL SEQ ID NO.: 42; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

43. A therapeutic composition comprising a S-CoV PODTC3 ‘ORF3A’ protein peptide, beginning at position 53 -69 of LAVFQSASKIITLKKRW SEQ ID NO.: 43; optionally in combination with one or more of MBL/mMBL, an adjuvant, a carrier, an additional therapeutic, and/or an excipient. The composition is optionally formulated for nasal/nasopharyngeal administration. The composition is optionally in the form of a nanoparticulate, such as an encapsulated nanoparticulate.

44. The therapeutic composition can comprise one or more peptides selected from the group consisting of SEQ ID 1-43, in combination with MBL/mMBL optionally in oligomeric form, wherein the carrier is selected from the group consisting of: diphtheria toxoid (DT), tetanus toxoid (TT), pertussin toxoid (PT), Dukoral (Cholera Toxoid-B), BCG-proteins, Pure Protein Derivative (PPD), Measles, Mumps, Rubella, Varicella viral proteins, recombinant-TT, recombinant—HepB, HPV-VLP, and as carrier protein, including any FDA approved commercial (GMP) produced protein(s) able to provide CD4 stimulation.

45. The therapeutic composition can comprise one or more peptides selected from the group consisting of SEQ ID 1-43, in combination with MBL/mMBL optionally in oligomeric form, wherein the carrier is a recombinant expressed immunogenic polypeptide larger than 3000 Daltons (˜>300 amino acids) and may be an immunoglobulin class protein.

46. The therapeutic composition can comprise one or more peptides selected from the group consisting of SEQ ID 1-43 and an MBL/mMBL oligomer, in further combination with a pharmaceutically acceptable carrier to effect viral neutralizing and competitively protective titers in the majority of hosts introduced via nasopharyngeal, or mucosal routes (incl. sublingually) to effect reduction of upper airway viral entry.

47. The therapeutic composition can comprise one or more peptides selected from the group consisting of SEQ ID 1-43 and inclusive of items listed in 44-46 wherein the pharmaceutically acceptable mixture comprises nanoparticles, of PLA/PLGA biopolymers that are deliverable by standard pharmaceutically approved nasal spray equipment.

48. The therapeutic composition comprising one or more peptides selected from the group consisting of SEQ ID 1-43 and items 44-47, wherein the pharmaceutically acceptable therapeutic composition comprises a suitably acceptable vehicle such as an nanoparticulate aerosol spray components include mixtures of one or more innate stimulating adjuvants comprising, but not limited to: nano-particulate manganous, or aluminum or calcium phosphates, diguanylate cyclic monophosphates, guanosine, (cyclic-di-GMP) or other cyclic-di mononucleotides that activate STING immunological pathways, Praziquantel, Imiquimod, Nor-MDP, Ergamisol, Cimetidine, uric acid, cyclic diguanylate, threonyl-N-acetyl-muramyl-L-alanyl-D-isoglutamine, Isoprinosine, mannan, trehalose dimycolate, QS-21 and alpha-galactosylceramide (α-GalCer) or alphaglucosylceramide (α-GluCer).

49. The therapeutic composition can comprise one or more peptides selected from the group consisting of SEQ ID 1-43, further in combination with a combination of antibodies against the respiratory virus of interest (e.g., monoclonal antibodies such as casirivimab and imdevimab), remdesivir, molnupiravir, AT-527, and/or PF-07321332, and optionally in combination with one or more of (i) MBL/mMBL optionally in oligomeric form, (ii) a carrier, (iii) an additional therapeutic and/or (iv) an excipient.

50. The therapeutic composition can comprise a MBL/mMBL, optionally in oligomeric form, in combination with a combination of antibodies against the respiratory virus of interest (e.g., monoclonal antibodies such as casirivimab and imdevimab), remdesivir, molnupiravir, AT-527, and/or PF-07321332, and optionally in combination with one or more of (i) one or more peptides selected from the group consisting of SEQ ID 1-43, (ii) a carrier, (iii) an additional therapeutic and/or (iv) an excipient.

The invention can elicit disease ameliorating and/or protective innate processes, including MBL assisted complement defenses and/or otherwise initiate and/or promote adaptive immunologic responses including promoting memory resident T-cell responses able to recognize non-mutated, highly conserved epitopes in the pathogenic protein regions and/or enable the glycosidic residue regions that may become accessible by MBL binding to the intact virion and thus can be more able to have complement remove pathogens/virions even when variant mutations arise in immunodominant epitopes that would allow antibody escape.

### Adjuvants

Non-limiting examples of adjuvants useful in any of the compositions described herein include nano-particulate manganous, or aluminum or calcium phosphates, diguanylate cyclic monophosphates, guanosine, (cyclic-di-GMP) or other cyclic-di mononucleotides that activate STING immunological pathways, Praziquantel, Imiquimod, Nor-MDP, Ergamisol, Cimetidine, uric acid, cyclic diguanylate, threonyl-N-acetyl-muramyl-L-alanyl-D-isoglutamine, Isoprinosine, mannan, trehalose dimycolate, QS-21 and alpha-galactosylceramide (α-GalCer) or alphaglucosylceramide (α-GluCer), and combinations thereof.

Additional non-limiting examples of adjuvants useful in any of the compositions described herein include toxoidal peptide(s) derived from, but not limited to: tetanus toxoid [TT]; diphtheria toxoid [DT]; pertussis toxoid [PT]; cholera toxoid [CT or Dukoral®]; or mixtures thereof.

### Additional Therapeutics

It is contemplated that when used to treat various diseases, the compositions and methods of the present invention can be combined with other therapeutic agents suitable for the same or similar diseases. Also, two or more embodiments of the invention may be also co-administered to generate additive or synergistic effects. When co-administered with a second therapeutic agent, the embodiment of the invention and the second therapeutic agent may be simultaneously or sequentially (in any order). Suitable therapeutically effective dosages for each agent may be lowered due to the additive action or synergy.

As a non-limiting example, the invention can be combined with other therapies that treat and/or prevent viral infections.

Non-limiting examples of useful anti-viral drugs include interferon alpha, beta or gamma, didanosine, lamivudine, zanamavir, lopanivir, nelfinavir, efavirenz, indinavir, valacyclovir, zidovudine, amantadine, rimantidine, ribavirin, ganciclovir, foscarnet, and acyclovir or any salts or variants thereof. See also Physician's Desk Reference, 59th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

Other non-limiting examples of useful antiviral drugs include (i) rilpivine, abacavir and lamivudine; (ii) abacavir, dolutegravir and lamivudine; (iii) abacavir, lamivudine and zidovudine; (iv) atazanavir and cobicistat; (v) darunavir and cobicistat; (vi) efavirenz, emtricitabine and tenofovir disoproxil fumerate; (vii) elvitegravir, cobicistat, emtricitabine, tenofovir alafenamide fumerate; (viii) elvitegravir, cobicistat, emtricitabine and tenofovir disoproxil fumerate; (ix) emtricitabine, rilpivirine and tenofovir alafenamide; (x) memtricitabine, rilpivirine and tenofovir disoproxil fumerate; (xi) emtricitabine and tenofovir alafenamide; (xii) emtricitabine and tenofovir disoproxil fumerate; (xiii) lamivudine and zidovudine; (xiv) lopinavir, retinovir and interferon-beta 1b; (xv) lopinavir and ritonavir, and (xvi) remdesivir, AT-527, molnupravir (EIDD-2801) and PF-07321332.

Preferably the antiviral agent comprises one or more of remdesivir, AT-527, molnupravir (EIDD-2801) and/or PF-07321332.

Antibodies against the respiratory virus of interest may also be included, such as monoclonal or polyclonal antibodies, and combinations thereof. A nonlimiting example of such antibodies is a combination of monoclonal antibodies casirivimab and imdevimab.

### Pharmaceutical Formulations and Administration

The compositions of the invention can comprise a carrier and/or excipient. While it is possible to use a composition of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Solid dosage forms for oral administration can also be used and can include, e.g., capsules, tablets, caplets, pills, troches, lozenges, powders, and granules. Non-limiting examples of suitable excipients include, e.g., diluents, buffering agents, preservatives, stabilizers, binders, compaction agents, lubricants, dispersion enhancers, disintegration agents, antioxidants, flavoring agents, sweeteners, and coloring agents. Those of relevant skill in the art are well able to prepare suitable solutions.

In one embodiment of any of the compositions of the invention, the composition is formulated for delivery by a route such as, e.g., oral, topical, rectal, mucosal, sublingual, nasal, naso/oro-gastric gavage, parenteral, intraperitoneal, intradermal, transdermal, intrathecal, nasal, nasopharyngeal, and intra-tracheal administration. In one embodiment of any of the compositions of the invention, the composition is in a form of a liquid, foam, cream, spray, powder, or gel.

Administration of the compounds and compositions in the methods of the invention can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, nasopharyngeal, and intra-tracheal administration. The active agent may be systemic after administration or may be localized by the use of regional administration, intramural administration, or use of an implant that acts to retain the active dose at the site of implantation.

The useful dosages of the compounds and formulations of the invention can vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses may be effective to achieve a therapeutic effect. While it is possible to use a compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice. Although there are no physical limitations to delivery of the formulations of the present invention, nasal or nasopharyngeal delivery is preferred for delivery to the nasal passages.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Oral pharmaceutical dosage forms include, by way of example and without limitation, solid, gel and liquid. Solid dosage forms include tablets, capsules, granules, lozenges, and bulk powders. Oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, lozenges, troches and the like can contain any of the following ingredients, or agents of a similar nature: a binder; a diluent; a disintegrating agent; a lubricant; a glidant; a sweetening agent; and a flavoring agent.

Examples of binders include, by way of example and without limitation, microcrystalline cellulose, gum tragacanth, glucose solution, acacia mucilage, gelatin solution, sucrose, and starch paste. Lubricants include, by way of example and without limitation, talc, starch, magnesium or calcium stearate, lycopodium and stearic acid. Diluents include, by way of example and without limitation, lactose, sucrose, starch, kaolin, salt, mannitol, and dicalcium phosphate. Glidants include, by way of example and without limitation, colloidal silicon dioxide. Disintegrating agents include, by way of example and without limitation, crosscarmellose sodium, sodium starch glycolate, alginic acid, corn starch, potato starch, bentonite, methylcellulose, agar and carboxymethylcellulose. Coloring agents include, by way of example and without limitation, any of the approved certified water soluble FD and C dyes, mixtures thereof; and water insoluble FD and C dyes suspended on alumina hydrate. Sweetening agents include, by way of example and without limitation, sucrose, lactose, mannitol and artificial sweetening agents such as saccharin, and any number of spray dried flavors. Flavoring agents include, by way of example and without limitation, natural flavors extracted from plants such as fruits and synthetic blends of agents which produce a pleasant sensation, such as, but not limited to peppermint and methyl salicylate. Wetting agents include, by way of example and without limitation, propylene glycol monostearate, sorbitan monooleate, diethylene glycol monolaurate, and polyoxyethylene laural ether. Emetic-coatings include, by way of example and without limitation, fatty acids, fats, waxes, shellac, ammoniated shellac and cellulose acetate phthalates. Film coatings include, by way of example and without limitation, hydroxyethylcellulose, sodium carboxymethylcellulose, polyethylene glycol 4000 and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents may be used in any of the above dosage forms.

Solvents include, by way of example and without limitation, glycerin, sorbitol, ethyl alcohol and syrup. Examples of preservatives include, without limitation, glycerin, methyl and propylparaben, benzoic acid, sodium benzoate and alcohol. Non-aqueous liquids utilized in emulsions include, by way of example and without limitation, mineral oil and cottonseed oil. Emulsifying agents include, by way of example and without limitation, gelatin, acacia, tragacanth, bentonite, and surfactants such as polyoxyethylene sorbitan monooleate. Suspending agents include, by way of example and without limitation, sodium carboxymethylcellulose, pectin, tragacanth, Veegum and acacia. Diluents include, by way of example and without limitation, lactose and sucrose. Sweetening agents include, by way of example and without limitation, sucrose, syrups, glycerin and artificial sweetening agents such as saccharin. Wetting agents include, by way of example and without limitation, propylene glycol monostearate, sorbitan monooleate, diethylene glycol monolaurate, and polyoxyethylene lauryl ether. Organic acids include, by way of example and without limitation, citric and tartaric acid. Sources of carbon dioxide include, by way of example and without limitation, sodium bicarbonate and sodium carbonate. Coloring agents include, by way of example and without limitation, any of the approved certified water soluble FD and C dyes, and mixtures thereof. Flavoring agents include, by way of example and without limitation, natural flavors extracted from plants, such as fruits, and synthetic blends of agents which produce a pleasant taste sensation.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution can be apportioned into vials for lyophilization. Each vial can contain, by way of example and without limitation, a single dosage (10-1000 mg, such as 100-500 mg) or multiple dosages of the agent. The lyophilized powder can be stored under appropriate conditions, such as at about 4° C. to room temperature. Reconstitution of this lyophilized powder with water for injection provides a formulation for use in parenteral administration. For reconstitution, about 1-50 mg, such as about 5-35 mg, for example, about 9-30 mg of lyophilized powder, is added per mL of sterile water or other suitable carrier. The precise amount depends upon the selected agent. Such amount can be empirically determined.

The inventive composition or pharmaceutically acceptable derivatives thereof may be formulated as aerosols for application e.g., by inhalation or intranasally (e.g., as described in U.S. Pat. No. 4,044,126, 4,414,209, and 4,364,923). These formulations can be in the form of an aerosol or solution for a nebulizer, or as a microfine powder for insufflation, alone or in combination with an inert carrier such as lactose. In such a case, the particles of the formulation can, by way of example and without limitation, have diameters of less than about 50 microns, such as less than about 10 microns.

The agents may be also formulated for local or topical application, such as for application to the skin and mucous membranes (e.g., intranasally), in the form of nasal solutions, gels, creams, and lotions.

### Prophylactic Applications

In some embodiments, SARS-CoV-2 agents in accordance with the invention may be utilized for prophylactic applications. In some embodiments, prophylactic applications involve systems and methods for preventing, inhibiting progression of, and/or delaying the onset of SARS-CoV-2 infection, and/or any other SARS-CoV-2 -associated condition in individuals susceptible to and/or displaying symptoms of SARS-CoV-2 infection. In some embodiments, prophylactic applications involve systems and methods for preventing, inhibiting progression of, and/or delaying the onset of infection of the brain. In some embodiments, prophylactic applications involve systems and methods for preventing, inhibiting progression of, and/or delaying the impairment of vital organs (e.g., liver).

The present formulations may be packaged for administration in any conventional manner, preferably in a nasal applicator, and preferably in such a way as to deliver a fixed dose of drug substance (e.g., active ingredient). However, the present formulations may be administered via a nasal application in such a way as to deliver a non-fixed dose of drug substance. Spray administration containers for various types of nasal formulations have been known in the past and substantially all will be equally suitable for the present formulations, considering of course that the materials from which the container is made is compatible with the formulations. The medium containing the drug substance and other appropriate ingredients may be contained in a small bottle or similar container, from which it can be dispersed as a mist to be directed into each nostril. Using ambient air as the propelling agent, one may have the bottle made of a flexible plastic, so that merely squeezing the bottle's sides impels the spray out through the nozzle into the nasal cavity. Air may also be the propelling agent for a pump sprayer, in which the user manipulates a small pump button which pumps air into the container and causes the liquid spray to be emitted on the return stroke. Alternatively, the bottle can be pressurized with a gas which is inert to the user and to the ingredients of the solution. The gas may be dissolved under pressure in the container or may be generated by dissolution or reaction of a solid material which forms the gas as a product of dissolution or as a reaction product. Typical gases which can be used include nitrogen, argon, and carbon dioxide. Also, when the formulation is administered as a spray or aerosol, the formulation may be contained in a pressurized container with a liquid propellant including, but not limited to dicholorodifluoro methane or chlorotrifluoro ethylene, among other propellants.

In another alternative embodiment, for administration as a spray, the present formulations may be placed in an appropriate atomizing device, e.g. in a pump-atomiser or the like. The atomizing device may be provided with appropriate means for delivery of aqueous spray to the naris. Preferably, it is provided with means ensuring delivery of a substantially fixed volume of composition/actuation (i.e. per spray-unit). In one embodiment, the device administers a metered dosage. The spray composition may be suspended or dissolved in a liquid propellant. Stabilizing and/or suspending agents and/or co-solvents may be present. In other embodiments herein, the formulation of the present invention is suitable for administration intranasally via a metered-dose spray pump to a subject in need thereof. In this respect, the formulation of the present invention may be pre-packaged in a metered-dose spray pump bottle, or metering atomizing pump.

In another alternative embodiment, the formulations of the present invention may be administered into the nose in the form of drops, or any other method which results in topical application to the nasal mucosa. The form of dosage for intranasal administration may include solutions, suspensions or emulsions of the active compound in a liquid carrier in the form of nose drops. Suitable liquid carriers include water, propylene glycol and other pharmaceutically acceptable alcohols. For administration in drop form formulations may suitably be put in a container provided e.g. with a conventional dropper/closure device, e.g. comprising a pipette or the like, preferably delivering a substantially fixed volume of composition/drop. The dosage forms may be sterilized, as required. The dosage forms may also contain adjuvants such as preservatives, stabilizers, emulsifiers or suspending agents, wetting agents, salts for varying the osmotic pressure or buffers, as required.

In another alternative embodiment, the present formulations may be administered in the form of a powder. For example, a powdery nasal composition can be directly used as a powder for a unit dosage form. If desired, the powder can be filled in capsules such as hard gelatin capsules. The contents of the capsule or single dose device may be administered using e.g. an insufflator. Preferably, it is provided with means ensuring dosing of a substantially fixed amount of composition/actuation.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Administration of the Therapeutic Compositions

A therapeutic composition as described herein can be administered in at least one dose. A second dose can be administered at a desired time after administration of the first dose. The amount of the disclosed MBL/mMBL and/or peptides can be the same in the first dose and the second dose, or the amounts can be different.

The therapeutic compositions can be administered by oral, nasal or nasopharyngeal administration, e.g., nasal sprays, drops, lotions, creams, foams, and the like, as well as a buccal spray, mouthwash, rapidly dissolving sublingual or buccal tablet, or rapidly dissolving thin film. The therapeutic composition may be in nanoparticle form, such as microencapsulated nanoparticles.

A therapeutic composition as described herein can be formulated in an amount of, e.g., 100 micrograms of an active agent of the invention (e.g., MBL/mMBL and/or peptide composition) per 1 ml of pharmaceutically acceptable excipients) for nasal delivery such that about 1 mL is delivered to the nasal mucosa per nostril of a patient in need thereof twice a day for a two-week period.

### List of Items

1. A method for treating or preventing SARS-COV-2 comprising administering to a patient in need thereof a therapeutically effective amount of a therapeutic composition comprising a mannose binding lectin (MBL)/modified mannose binding lectin (mMBL) and mixtures thereof, optionally in further combination with one or more of an adjuvant, a carrier, and an excipient.

2. The method according to item 1, wherein the MBL/mMBL is an oligomer comprising at least one MBL subunit.

3. The method according to item 2, wherein the MBL/mMBL oligomer is a tetramer, pentamer or hexamer

4. The method according to item 3, wherein the MBL oligomer is a tetramer or hexamer

5. The method according to any of items 1-4, further comprising an adjuvant.

6. The method according to item 5, wherein the adjuvant exhibits a mucosal IgA response.

7. The method according to item 5, wherein the adjuvant is a linear peptide of the SARS-COV-2 virus selected from the group consisting of SEQ ID 1-43 that exhibits a mucosal IgA response.

8. The method according to any of items 1-7, wherein the therapeutic composition comprises between about 1 and about 1000 μg/mL of an MBL/mMBL.

9. The method according to item 8, wherein the therapeutic composition comprises between about 1 and about 100 μg/mL of the MBL/mMBL.

10. The method according to any of items 1-9, wherein the therapeutic composition is administered in a form selected from an oral, oral mucosal, nasal, or nasopharyngeal formulation.

11. The method according to any of items 1-9, wherein the therapeutic composition is in a form selected from an oil suspension, a spray, a suspension, a solution, an emulsion, a nasal spray, nasal drops, a buccal spray, nasal rinse, nasal cream, nasal gel, mouthwash, rapidly dissolving sublingual or buccal tablet, rapidly dissolving thin film, and formulations adapted for inhalation, insufflation, and/or nebulization.

12. The method according to item 11, wherein the pharmaceutical formulation is in the form of a nasal spray.

13. The method according to any of items 1-12, wherein the pharmaceutical formulation is contained in an atomizer or inhaler.

14. The method according to any of items 1-13, wherein the pharmaceutical formulation further comprises alkali or alkali earth metal ions.

15. The method according to item 14, wherein the alkali earth metal ion is Ca++.

16. The method according to any of items 1-15, further comprising the administration of an antiviral agent chosen from the group consisting of an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, and an antibody therapy (monoclonal or polyclonal) or combination thereof.

17. The method according to item 16, wherein the antiviral agent is chosen from the group consisting of remdesivir, AT-527, molnupravir (EIDD-2801) and PF-07321332.

18. The method according to item 16, wherein a combination of antiviral agents is chosen from the group consisting of (i) rilpivine, abacavir and lamivudine; (ii) abacavir, dolutegravir and lamivudine; (iii) abacavir, lamivudine and zidovudine; (iv) atazanavir and cobicistat; (v) darunavir and cobicistat; (vi) efavirenz, emtricitabine and tenofovir disoproxil fumerate; (vii) elvitegravir, cobicistat, emtricitabine, tenofovir alafenamide fumerate; (viii) elvitegravir, cobicistat, emtricitabine and tenofovir disoproxil fumerate; (ix) emtricitabine, rilpivirine and tenofovir alafenamide; (x) memtricitabine, rilpivirine and tenofovir disoproxil fumerate; (xi) emtricitabine and tenofovir alafenamide; (xii) emtricitabine and tenofovir disoproxil fumerate; (xiii) lamivudine and zidovudine; (xiv) lopinavir, retinovir and interferon-beta 1b; (xv) lopinavir and ritonavir, and (xvi) remdesivir, AT-527, molnupravir (EIDD-2801) and PF-07321332.

19. The method according to item 16, wherein the antiviral agent is a combination of remdesivir, AT-527, molnupravir (EIDD-2801) and/or PF-07321332.

20. The method according to any of items 1-19 further comprising a combination of antibodies against the respiratory virus of interest (e.g., monoclonal antibodies such as casirivimab and imdevimab).

21. The method according to any of items 1-20, wherein the MBL/mMBL and/or peptide component is microencapsulated.

22. The method according to any of items 1-20, wherein the MBL/mMBL and/or peptide component is formulated or co-formulated in nanoparticles, preferably microencapsulated nanoparticles.

23. A method for treating or preventing SARS-COV-2 comprising administering to a patient in need thereof a therapeutically effective amount of a therapeutic composition comprising a mannose binding lectin (MBL)/modified mannose binding lectin (mMBL) and mixtures thereof, in combination with at least one peptide selected from the group consisting of SEQ ID NOs: 1-43, optionally in further combination with one or more of an adjuvant, a carrier, and an excipient.

24. The method according to item 23, wherein the therapeutic composition comprises between about 1 and about 1000 μg/mL of MBL/mMBL.

25. The method according to items 23-24, wherein the therapeutic composition comprises between about 1 and about 100 μg/mL of MBL/mMBL.

26. The method according to any of items 23-25, wherein the therapeutic composition is administered in the form of an oral, oral mucosal, nasal, or nasopharyngeal formulation.

27. The method according to any of items 23-26, wherein the therapeutic composition is in a form selected from an oil suspension, a spray, a suspension, a solution, an emulsion, a nasal spray, nasal drops, a buccal spray, nasal rinse, nasal cream, nasal gel, mouthwash, rapidly dissolving sublingual or buccal tablet, rapidly dissolving thin film, and formulations adapted for inhalation, insufflation, and/or nebulization.

28. The method according to any of items 23-27, wherein the therapeutic composition is in the form of a nasal spray.

29. The method according to any of items 23-28, wherein the therapeutic composition is contained in an atomizer or inhaler.

30. The method according to any of items 23-29, wherein the therapeutic composition further comprises alkali or alkali earth metal ions.

31. The method according to item 30, wherein the alkali earth metal ion is Ca++.

32. The method according to any of items 23-31 further comprising the administration of an antiviral agent chosen from the group consisting of an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, and an antibody therapy (monoclonal or polyclonal) or combination thereof.

33. The method according to item 32, wherein the antiviral agent is chosen from the group consisting of remdesivir, AT-527, molnupravir (EIDD-2801) and PF-07321332.

34. The method according to item 32, wherein a combination of antiviral agents is chosen from the group consisting of (i) rilpivine, abacavir and lamivudine; (ii) abacavir, dolutegravir and lamivudine; (iii) abacavir, lamivudine and zidovudine; (iv) atazanavir and cobicistat; (v) darunavir and cobicistat; (vi) efavirenz, emtricitabine and tenofovir disoproxil fumerate; (vii) elvitegravir, cobicistat, emtricitabine, tenofovir alafenamide fumerate; (viii) elvitegravir, cobicistat, emtricitabine and tenofovir disoproxil fumerate; (ix) emtricitabine, rilpivirine and tenofovir alafenamide; (x) memtricitabine, rilpivirine and tenofovir disoproxil fumerate; (xi) emtricitabine and tenofovir alafenamide; (xii) emtricitabine and tenofovir disoproxil fumerate; (xiii) lamivudine and zidovudine; (xiv) lopinavir, retinovir and interferon-beta 1b; (xv) lopinavir and ritonavir, and (xvi) remdesivir, AT-527, molnupravir (EIDD-2801) and PF-07321332.

35. The method according to item 32, wherein the antiviral agent is a combination of remdesivir, AT-527, molnupravir (EIDD-2801) and/or PF-07321332.

36. The method according to any of items 23-35 further comprising a combination of antibodies against the respiratory virus of interest (e.g., monoclonal antibodies such as casirivimab and imdevimab).

37. The method according to any of items 23-36, wherein the MBL/mMBL is microencapsulated.

38. The method according to item 23 wherein the MBL/mMBL is formulated or co-formulated in nanoparticles, preferably microencapsulated nanoparticles.

39. A method for treating or preventing SARS-COV-2 comprising administering to a patient in need thereof a therapeutically effective amount of a therapeutic composition comprising at least one peptide selected from the group consisting of SEQ ID NOs: 1-43.

40. The method according to item 39, wherein the therapeutic composition further comprises at least three peptides selected from the group consisting of SEQ ID NOs: 1-43.

41. The method according to items 39-40 further comprising an MBL/mMBL.

42. The method according to item 41, wherein the MBL is a tetramer, pentamer or hexamer

43. The method according to item 42, wherein the MBL is a tetramer or a hexamer.

44. The method according to items 39-43, wherein the therapeutic composition further comprises one or more of an adjuvant, a carrier, and an excipient.

45. The method according to item 44, wherein the adjuvant exhibits a mucosal IgA response.

46. The method according to item 44, wherein the adjuvant comprises a toxoidal peptide(s) derived from, but not limited to: tetanus toxoid [TT]; diphtheria toxoid [DT]; pertussis toxoid [PT]; cholera toxoid [CT or Dukoral®]; or mixtures thereof.

47. The method according to item 41, wherein the therapeutic composition comprises between about 1 and about 1000 μg/mL of an MBL/mMBL.

48. The method according to item 47, wherein the therapeutic composition comprises between about 1 and about 100 μg/mL of an MBL/mMBL.

49. The method according to any of items 39-48, wherein the therapeutic composition is administered as in the form of an oral, oral mucosal, nasal, or nasopharyngeal formulation.

50. The method according to any of items 39-49, wherein the therapeutic composition is in a form selected from an oil suspension, a spray, a suspension, a solution, an emulsion, a nasal spray, nasal drops, a buccal spray, nasal rinse, nasal cream, nasal gel, mouthwash, rapidly dissolving sublingual or buccal tablet, rapidly dissolving thin film, and formulations adapted for inhalation, insufflation, and/or nebulization.

51. The method according to item 50, wherein the therapeutic composition is in the form of a nasal spray.

52. The method according to any of items 39-51, wherein the therapeutic composition is contained in an atomizer or inhaler.

53. The method according to any of items 39-52, wherein the therapeutic composition further comprises alkali or alkali earth metal ions.

54. The method according to item 53, wherein the alkali earth metal ion is Ca++.

55. The method according to any of items 39-54 further comprising the administration of an antiviral agent chosen from the group consisting of an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, and an antibody therapy (monoclonal or polyclonal) or combination thereof.

56. The method according to item 55, wherein the antiviral agent is chosen from the group consisting of remdesivir, AT-527, molnupravir (EIDD-2801) and PF-07321332.

57. The method according to item 55, wherein a combination of antiviral agents is chosen from the group consisting of (i) rilpivine, abacavir and lamivudine; (ii) abacavir, dolutegravir and lamivudine; (iii) abacavir, lamivudine and zidovudine; (iv) atazanavir and cobicistat; (v) darunavir and cobicistat; (vi) efavirenz, emtricitabine and tenofovir disoproxil fumerate; (vii) elvitegravir, cobicistat, emtricitabine, tenofovir alafenamide fumerate; (viii) elvitegravir, cobicistat, emtricitabine and tenofovir disoproxil fumerate; (ix) emtricitabine, rilpivirine and tenofovir alafenamide; (x) memtricitabine, rilpivirine and tenofovir disoproxil fumerate; (xi) emtricitabine and tenofovir alafenamide; (xii) emtricitabine and tenofovir disoproxil fumerate; (xiii) lamivudine and zidovudine; (xiv) lopinavir, retinovir and interferon-beta 1b; (xv) lopinavir and ritonavir, and (xvi) remdesivir, AT-527, molnupravir (EIDD-2801) and PF-07321332.

58. The method according to item 55, wherein the anti-viral agent is a combiantio of remdesivir, AT-527, molnupravir (EIDD-2801) and/or PF-07321332.

59. The method according to any of items 41-54 further comprising a combination of antibodies against the respiratory virus of interest (e.g., monoclonal antibodies such as casirivimab and imdevimab).

60. The method according to any of items 41-59, wherein the MBL/mMBL is microencapsulated.

61. The method according to any of items 41-60, wherein the MBL/mMBL is formulated or co-formulated in nanoparticles.

62. The method according to any of items 1-20, wherein the composition comprises MBL/mMBL and/or at least one peptide selected from the group consisting of SEQ ID NOs: 1-43, optionally in further combination with a carrier, an adjuvant, an excipient, and/or a combination of antibodies against the respiratory virus of interest (e.g., monoclonal antibodies such as casirivimab and imdevimab), remdesivir, AT-527, molnupiravir and/or PF-07321332, and optionally in a nasal, oral, or nasopharyngeal formulation.

63. The method according to any of items 21-38, wherein the composition comprises MBL/mMBL and/or at least one peptide selected from the group consisting of SEQ ID NOs: 1-43, optionally in further combination with a carrier, an adjuvant, an excipient, and/or a combination of antibodies against the respiratory virus of interest (e.g., monoclonal antibodies such as casirivimab and imdevimab), remdesivir, AT-527, molnupiravir and/or PF-07321332, and optionally in a nasal, oral, or nasopharyngeal formulation.

64. The method according to any of items 39-61, wherein the composition comprises MBL/mMBL and/or at least one peptide selected from the group consisting of SEQ ID NOs: 1-43, optionally in further combination with a carrier, an adjuvant, an excipient, and/or a combination of antibodies against the respiratory virus of interest (e.g., monoclonal antibodies such as casirivimab and imdevimab), remdesivir, AT-527, molnupiravir and/or PF-07321332, and optionally in a nasal, oral, or nasopharyngeal formulation.

65. A method for method that simultaneously stimulates both the innate and adaptive immune responses against a respiratory virus and opsonizes its elimination by the proximal aggregation of the invading virus to its targeted mucosal antibody, comprising administration of a therapeutic composition comprising:
a MBL/mMBL;
peptides that induce an immune response to the respiratory virus; and
optionally one or more of an adjuvant, carrier, excipient, and an additional therapeutic.

66. The method according to item 65, wherein the therapeutic composition comprises at least three peptides selected from the group consisting of SEQ ID NOs: 1-43.

67. The method according to items 65-66 wherein the MBL is a tetramer, pentamer or hexamer

68. The method according to item 67, wherein the MBL is a tetramer or a hexamer.

69. The method according to items 65-68, wherein the therapeutic composition further comprises one or more of an adjuvant, a carrier, and an excipient.

70. The method according to item 69, wherein the adjuvant exhibits a mucosal IgA response.

71. The method according to item 70, wherein the adjuvant comprises a toxoidal peptide(s) derived from, but not limited to: tetanus toxoid [TT]; diphtheria toxoid [DT]; pertussis toxoid [PT]; cholera toxoid [CT or Dukoral®]; or mixtures thereof.

72. The method according to any of items 65-71, wherein the therapeutic composition comprises between about 1 and about 1000 μg/mL of an MBL/mMBL.

73. The method according to any of items 65-71, wherein the therapeutic composition comprises between about 1 and about 100 μg/mL of an MBL/mBL.

74. The method according to any of items 65-73, wherein the therapeutic composition is administered as in the form of an oral, oral mucosal, nasal, or nasopharyngeal formulation.

75. The method according to any of items 39-49, wherein the therapeutic composition is in a form selected from an oil suspension, a spray, a suspension, a solution, an emulsion, a nasal spray, nasal drops, a buccal spray, nasal rinse, nasal cream, nasal gel, mouthwash, rapidly dissolving sublingual or buccal tablet, rapidly dissolving thin film, and formulations adapted for inhalation, insufflation, and/or nebulization.

76. The method according to item 75, wherein the therapeutic composition is in the form of a nasal spray.

77. The method according to any of items 65-76, wherein the therapeutic composition is contained in an atomizer or inhaler.

78. The method according to any of items 65-77, wherein the therapeutic composition further comprises alkali or alkali earth metal ions.

79. The method according to item 78, wherein the alkali earth metal ion is Ca++.

80. The method according to any of items 65-79 further comprising the administration of an antiviral agent chosen from the group consisting of an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, and an antibody therapy (monoclonal or polyclonal) or combination thereof.

81. The method according to item 80, wherein the antiviral agent is chosen from the group consisting of remdesivir, AT-527, molnupravir (EIDD-2801) and PF-07321332.

82. The method according to item 80, wherein a combination of antiviral agents is chosen from the group consisting of (i) rilpivine, abacavir and lamivudine; (ii) abacavir, dolutegravir and lamivudine; (iii) abacavir, lamivudine and zidovudine; (iv) atazanavir and cobicistat; (v) darunavir and cobicistat; (vi) efavirenz, emtricitabine and tenofovir disoproxil fumerate; (vii) elvitegravir, cobicistat, emtricitabine, tenofovir alafenamide fumerate; (viii) elvitegravir, cobicistat, emtricitabine and tenofovir disoproxil fumerate; (ix) emtricitabine, rilpivirine and tenofovir alafenamide; (x) memtricitabine, rilpivirine and tenofovir disoproxil fumerate; (xi) emtricitabine and tenofovir alafenamide; (xii) emtricitabine and tenofovir disoproxil fumerate; (xiii) lamivudine and zidovudine; (xiv) lopinavir, retinovir and interferon-beta 1b; (xv) lopinavir and ritonavir, and (xvi) remdesivir, AT-527, molnupravir (EIDD-2801) and PF-07321332.

83. The method according to item 80, wherein the anti-viral agent is a combination of remdesivir, AT-527, molnupravir (EIDD-2801) and/or PF-07321332.

84. The method according to any of items 85-83 further comprising a combination of antibodies against the respiratory virus of interest (e.g., monoclonal antibodies such as casirivimab and imdevimab).

85. The method according to any of items 65-84, wherein the MBL/mMBL is microencapsulated.

86. The method according to any of items 65-85, wherein the MBL/mMBL is formulated or co-formulated in nanoparticles.

87. The method according to item 86, wherein the MBL is a tetramer, pentamer or hexamer

88. The method according to item 87, wherein the MBL is a tetramer or a hexamer.

89. The method according to any of items 65-88, wherein the therapeutic composition comprises MBL/mMBL and/or at least one peptide selected from the group consisting of SEQ ID NOs: 1-43, optionally in further combination with a carrier, an adjuvant, an excipient, and/or a combination of antibodies against the respiratory virus of interest (e.g., monoclonal antibodies such as casirivimab and imdevimab), remdesivir, AT-527, molnupiravir and/or PF-07321332, and optionally in a nasal, oral, or nasopharyngeal formulation.

### Sequence Listing

All sequences are amino acid sequences and all are artificial.

| SEQ ID | |
|---|---|
| NO. | Sequence |
| 1 | YDPLQPELDSFKEELDKYFK |
| 2 | YDPLQPELDS |
| 3 | DSFKEELDKYFK |
| 4 | NATRFASVYAWNRKR |
| 5 | CASYQTQTNSPRRAR |
| 6 | VGGNYNYLYRLFRKS |
| 7 | YNYLYRLFRKSNLKP |
| 8 | VYDPLQPELDSFKEE |
| 9 | CCSCGSCCKFDEDDS |
| 10 | AGCLIGAEHVNNSYE |
| 11 | DPLQPELDSFKEELD |
| 12 | SCGSCCKFDEDDSEP |
| 13 | SFTRGVYYPDKVFRS |
| 14 | GTHWFVTQRNFYEPQ |
| 15 | DVVIGIVNNTVYDPL |
| 16 | CLIGAEHVNNSYECD |
| 17 | NCDVVIGIVNNTVYD |
| 18 | IDGSSGVVNPVMEPIYDEPTTTTSV |
| 19 | SGVVNPVMEP |
| 20 | IHFYSKWYIRVGARKSAP |
| 21 | KLGSLVVRCSFYEDFLEYHDVRVVL |
| 22 | MADSNGTITVEELKKLLEQWN |
| 23 | MADSNGTITVE |
| 24 | MWLSYFIASFRLFARTRSM |
| 25 | NAAIVLQLPQGTTLPKGFYA |
| 26 | HIDAYKTFPPTEPKKDKKKKA |
| 27 | HYTPSFKKGAKLLHK |
| 28 | LHKPIVWHVNNATNKATYKPNTWCIR |
| 29 | CLWSTKPVE |
| 30 | GIVAIVVTCLAYYFMRFRRA |
| 31 | KHFYWFFSNYLKRRVVFNG |
| 32 | GVKHVYQLRARSVSPKLFIRQ |
| 33 | GYYRRATRRIR |
| 34 | SSPDDQIGYYRRATRRIR |
| 35 | DAYKTFPPTEPKKDKKKKADETQALPQRQKKQQTVTLLPAADLDD |
| 36 | SKOLQQSMSSSADS |
| 37 | VNNSYECDIPIGAGICASYQTQTNSPRRAR |
| 38 | NATRFASVYAWNRKRISNCVADYSLYN |
| 39 | SNNLDSKVGGNYNYLYRLFRKSNLKPFERDISTEIY |
| 40 | LNRALTGIAVEQDKNTEVFAQVKQIYKTPPIKDFGGFNFSQILPDPS |
| 41 | AYCCNIVNVSLVK |
| 42 | QIGGYTEKWESGVKDCVVL |
| 43 | LAVFQSASKIITLKKRW |

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1.-22. (canceled)

23. A method for treating or preventing SARS-COV-2 comprising administering to a patient in need thereof a therapeutically effective amount of a therapeutic composition comprising a mannose binding lectin (MBL)/modified mannose binding lectin (mMBL) and mixtures thereof, in combination with at least one peptide selected from the group consisting of SEQ ID NOs: 1-43, optionally in further combination with one or more of an adjuvant, a carrier, and an excipient.

24. The method according to claim 23, wherein the therapeutic composition comprises between about 1 and about 1000 μg/mL of MBL/mMBL.

25. The method according to claim 24, wherein the therapeutic composition comprises between about 1 and about 100 μg/mL of MBL/mMBL.

26. The method according to any of claim 23, wherein the therapeutic composition is administered in the form of an oral, oral mucosal, nasal, or nasopharyngeal formulation.

27. The method according to any of claim 26, wherein the therapeutic composition is in a form selected from an oil suspension, a spray, a suspension, a solution, an emulsion, a nasal spray, nasal drops, a buccal spray, nasal rinse, nasal cream, nasal gel, mouthwash, rapidly dissolving sublingual or buccal tablet, rapidly dissolving thin film, and formulations adapted for inhalation, insufflation, and/or nebulization.

28. The method according to any of claim 27, wherein the therapeutic composition is in the form of a nasal spray.

29. The method according to any of claim 27, wherein the therapeutic composition is contained in an atomizer or inhaler.

30. The method according to any of claim 23, wherein the therapeutic composition further comprises alkali or alkali earth metal ions.

31. The method according to claim 30, wherein the alkali earth metal ion is Ca++.

32. The method according to any of claim 23 further comprising the administration of an antiviral agent chosen from the group consisting of an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and combinations thereof.

33. The method according to claim 32, wherein the antiviral agent is chosen from the group consisting of remdesivir, AT-527, molnupravir (EIDD-2801) and PF-07321332.

34. The method according to claim 32, wherein a combination of antiviral agents is chosen from the group consisting of (i) rilpivine, abacavir and lamivudine; (ii) abacavir, dolutegravir and lamivudine; (iii) abacavir, lamivudine and zidovudine; (iv) atazanavir and cobicistat; (v) darunavir and cobicistat; (vi) efavirenz, emtricitabine and tenofovir disoproxil fumerate; (vii) elvitegravir, cobicistat, emtricitabine, tenofovir alafenamide fumerate; (viii) elvitegravir, cobicistat, emtricitabine and tenofovir disoproxil fumerate; (ix) emtricitabine, rilpivirine and tenofovir alafenamide; (x) memtricitabine, rilpivirine and tenofovir disoproxil fumerate; (xi) emtricitabine and tenofovir alafenamide; (xii) emtricitabine and tenofovir disoproxil fumerate; (xiii) lamivudine and zidovudine; (xiv) lopinavir, retinovir and interferon-beta 1b; (xv) lopinavir and ritonavir, and (xvi) remdesivir, AT-527, molnupravir (EIDD-2801) and PF-07321332.

35. The method according to claim 32, wherein the antiviral agent is a combination of remdesivir, AT-527, molnupravir (EIDD-2801) and/or PF-07321332.

36. The method according to claim 23 further comprising a combination of antibodies against the respiratory virus of interest (e.g., monoclonal antibodies such as casirivimab and imdevimab).

37. The method according to claim 23, wherein the MBL/mMBL is microencapsulated.

38. The method according to claim 23 wherein the MBL/mMBL is formulated or co-formulated in nanoparticles, preferably microencapsulated nanoparticles.

39. A method for treating or preventing SARS-COV-2 comprising administering to a patient in need thereof a therapeutically effective amount of a therapeutic composition comprising at least one peptide selected from the group consisting of SEQ ID NOs: 1-43.

40. The method according to claim 39, wherein the therapeutic composition further comprises at least three peptides selected from the group consisting of SEQ ID NOs: 1-43.

41. The method according to claim 39 further comprising an MBL/mMBL.

42. The method according to claim 41, wherein the MBL is a tetramer, pentamer or hexamer.

43-89. (canceled)
